(19) United States
(12) Patent Application Publication
(10) Pub. No.: US 2010/0036093 A1
(43) Pub. Date: Feb. 11, 2010

(54) CHOLESTEROLAMINE-INTRODUCED POLY-GAMMA-GLUTAMIC ACID DERIVATIVE

(75) Inventors: Hiroshi Uyama, Suita-shi (JP); Yasushi Osanai, Osaka-shi (JP); Moon-hee Sung, Daejeon (KR)

Correspondence Address: SEED INTELLECTUAL PROPERTY LAW GROUP PLLC, 701 FIFTH AVE, SUITE 5400, SEATTLE, WA, 98104, US

(73) Assignees: OSAKA UNIVERSITY, Suita-shi, Osaka (JP); GENOLAC BL CORPORATION, Ibaraki-shi, Osaka (JP); BIOLEADERS CORPORATION, Daejeon (KR)

(21) Appl. No.: 12/300,001

(22) PCT Filed: May 9, 2007
(86) PCT No.: PCT/JP2007/059625

§ 371 Date: Apr. 14, 2009

(30) Foreign Application Priority Data
May 9, 2006 (JP) 2006-130885

(51) Int. Cl. C07K 1/10

(52) U.S. Cl. 530/345

(57) ABSTRACT

Provided is a poly-γ-glutamic acid derivative having appropriately controlled hydrophilicity, which can be processed into a form of fine particles. Provided is a PGA derivative in a form of fine particles having a particle diameter of 50 to 1,000 nm, which is obtained by introducing cholesterol into poly-γ-glutamic acid (PGA). Thus, the PGA particles can be mixed with various biological materials (e.g., medicament) so as to be used in various applications in the field of biotechnology. Specifically, it can be used as a carrier of a medicament, or the like. Further, it is also made possible to use PGA in various applications in the field of nanotechnology that is studied in recent years.

### TECHNICAL FIELD

The present invention relates to a derivative of poly-γ-glutamic acid. More specifically, the present invention relates to a poly-γ-glutamic acid derivative in which cholesterol or its analogue is introduced.

### BACKGROUND ART

Poly-γ-glutamic acid, which is excellent in biodegradability and biocompatibility and also has moisture-retaining property, is attracting attention as a material for cosmetics, foods, and the like. Hereinafter in the present specification, poly-γ-glutamic acid will be also described as “PGA”.

Japanese Laid-open Patent Publication No. 2002-233391 (Patent Document 1) discloses a Bacillus strain producing a PGA having a high molecular weight.

International Publication WO 2004-7593 (Patent Document 2) discloses a PGA having an average molecular weight of 5,000 kDa or more.

PGA is characterized by being constituted with glutamic acid, and thus has high biocompatibility and has a possibility to be used as various materials in biotechnology. For that reason, it is desirable that PGA is mixed with various biological materials (e.g., medicaments), for use in various biotechnological applications. Further, if PGA can be processed into the form of a fine particle, it is made possible to use PGA in various applications in the field of nanotechnology, which has been studied in recent years.

However, PGA has many carboxyl groups in its molecular structure and is thus characterized in that the hydrophilicity is very high. Because of its excessively high hydrophilicity, PGA has a disadvantage that it cannot be processed into a form of a fine particle. It is hard to mix with a hydrophobic material. This disadvantage made it difficult to use PGA as a carrier of a medicament. It was also difficult to use PGA for various applications in the field of nanotechnology.

Further, Ishi-I, et al. (Non-patent Document 1) discloses a method of synthesizing a porphyrin cholesterol derivative and a method of introducing a primary amine into cholesterol by reacting cholesterol chloroformate with diamine.

A PGA derivative having introduced phenylalanine as a hydrophobic molecule is disclosed by Akagi, et al. (Non-patent Document 2). However, since phenylalanine does not have so high hydrophobicity, PGA can not be formed into a fine particle unless phenylalanine is introduced in a monomer unit ratio of 50% or more. Fine particles obtained by introducing a great amount of hydrophobic molecules into PGA that is a hydrophilic polymer has difficulty in sufficiently exerting performance inherent to PGA, and may be packed too densely because of the interaction among the hydrophobic groups introduced in a large amount.

In addition, in the field of creating medicaments, there is a need for a technique of making it possible to dissolve a medical agent that shows high pharmaceutical efficacy but is hardly soluble in water because of its excessively high hydrophobicity. Thus, there is a need for a hydrophilic polymer excellent in biocompatibility, which has the above described appropriate hydrophobic substituents. However, PGA is too hydrophilic as described above and often can not be used in such an application.

Regarding a carrier of a hydrophobic medical agent, for example, a technique of using a surfactant and the like are known (Patent Document 3).

Patent Document 1: Japanese Laid-open Patent Publication No. 2002-233391

Patent Document 2: International Publication WO 2004-7593

Patent Document 3: Japanese National Phase Laid-open Publication No. 10-503750

Non-patent Document 1: Langmuir, 2001, 17, pp. 5825-5833, [60] Fullerene Can Reinforce the Organogel Structure of Porphyrin-Appended Cholesterol Derivatives: Novel Odd-Even Effect of the (CH2)n Spacer on the Organogel Stability, Ishi-i et al.

Non-patent Document 2: Biomacromolecules, 2006, 7, pp. 297-303, Hydrolytic and Enzymatic Degradation of Nanoparticles Based on Amphiphilic Poly(γ-glutamic acid)-graft-L-Phenylalanine Copolymers, Akagi et al.

### DISCLOSURE OF THE INVENTION

### Problems to be Solved by the Invention

An object of the present invention is to solve the above problems and is to provide a PGA derivative having high biocompatibility and having fine particle forming ability by controlling the hydrophilicity of PGA having a peptide skeleton.

### Means for Solving the Problems

The inventors of the present invention repeated intensive studies, as a result, they have found that by introducing a trace amount of a derivative of cholesterol or its analogue having a primary amino group such as cholesterolamine, which is obtained as a derivative precursor, into PGA, a PGA derivative having appropriate hydrophilicity-hydrophobicity balance and having fine particle forming ability is provided, and based on the finding, the present invention was completed.

Specifically, the present invention provides the following PGA derivatives and others.

(1) A poly-γ-glutamic acid derivative, wherein cholesterol or its analogue is bound to the poly-γ-glutamic acid via a linker.

(2) The poly-γ-glutamic acid derivative according to item 1, which is represented by the following Formula 1:

R¹—NH—R³—NHC(═O)—O—R² Formula 1

wherein R¹ represents a residue of the poly-γ-glutamic acid, —O—R² represents a residue of the cholesterol or its analogue, and R³ represents alkylene.

(3) The poly-γ-glutamic acid derivative according to item 1, wherein a weight-average molecular weight of the poly-γ-glutamic acid is 1,000,000 or more.

(4) The poly-γ-glutamic acid derivative according to item 1, wherein the cholesterol or its analogue is bound to 0.1% to 10% of the side-chain carboxyl groups of the poly-γ-glutamic acid.

(5) A fine particle having an average diameter of 50 nm to 1000 nm, consisting of the poly-γ-glutamic acid derivative according to item 1.

(6) A method for producing the fine particle according to item 5, comprising a step of introducing cholesterol or its analogue into poly-γ-glutamic acid.

(7) A molecular chaperone for refolding a protein in a denatured state, consisting of the poly-γ-glutamic acid derivative according to item 1.

(8) A molecular chaperone for refolding a denatured protein in an aggregation state, consisting of the poly-γ-glutamic acid derivative according to item 1.

(9) A sustained release carrier, consisting of the poly-γ-glutamic acid derivative according to item 1.

### EFFECT OF THE INVENTION

According to the present invention, it is made possible to process PGA into a form of a fine particle. Specifically, the PGA derivative having a trace amount of introduced cholesterol or its analogue gives fine particles applicable to nanotechnology. Thus, the PGA particles can be mixed with various biological materials (e.g., medicament) so as to be used in various applications in biotechnology. Thus, it can be used as a carrier for medicaments. Further, it is also made possible to use PGA in various applications in the field of nanotechnology that is studied in recent years.

### BRIEF DESCRIPTION OF THE DRAWINGS

FIG. 1 shows the results of particle diameter measurement. The value of mol % in the box indicates a cholesterolamine (CHAm) introduction ratio.

FIG. 2 shows the results of particle-diameter stability measurement, indicating that the fine particles are present stably over an extended period of time.

FIG. 3 shows the results of determining the viscosity of the aqueous solutions of the derivatives, indicating that the solution was gelled when the viscosity is high.

FIG. 4 is SEM (scanning electron microscope) images of the particles obtained, confirming the presence of particles having a diameter of several hundred nm. The straight line below the left image corresponds to a length of 5 μm. The straight line below the right image corresponds to a length of 2 μm.

FIG. 5 shows the results of the evaluation of the sustained release properties of the PGA derivative according to the present invention. The line plotted on black square symbols indicates the results obtained in tests without adding calcium chloride. The line plotted on black circle symbols indicates the results obtained in the test in which 3.2 mM of calcium chloride was added at the time point of 3 hours. The line plotted on black triangle symbols indicates the results obtained in the test in which 20 mM of calcium chloride was added at the time point of 3 hours.

FIG. 6 shows the results obtained by evaluation of the efficiency of refolding a denatured enzyme by the PGA derivative according to the present invention. The line plotted on black square symbols indicates the results obtained in the tests by using PGA sodium salt (PGANa). The line plotted on black circle symbols indicates the results obtained in the tests of Example 14 using the derivative of Example 5. The line plotted on black triangle symbols indicates the results obtained in the tests of Example 15 using the derivative of Example 8.

FIG. 7 shows the results obtained by evaluation of the efficiency of refolding an aggregated enzyme by the PGA derivative according to the present invention. The line plotted on white square symbols indicates the results obtained in the tests of Example 16 using the derivative of Example 8. The line plotted on black triangle symbols indicates the results obtained in the test of Example 15 using a non-aggregated enzyme for the purpose of comparison.

### BEST MODE FOR CARRYING OUT THE INVENTION

Hereinafter, the present invention will be described in detail.

### (PGA)

Poly-γ-glutamic acid (PGA) is a compound obtained by a polymerization reaction of an amino group and a carboxyl group at the γ-position of D,L-glutamic acid. In the present invention, PGA may be also used in a form of its salt. For example, a sodium salt of PGA can be favorably used.

The molecular weight of PGA is not particularly limited. However, the weight-average molecular weight is preferably 10,000 or more, more preferably 30,000 or more, and further more preferably 50,000 or more, from the viewpoint of its physical properties. In embodiments where a high molecular weight PGA is desirable, a high molecular weight PGA having a weight-average molecular weight of 100,000 or more, 1,000,000 or more, or 2,000,000 or more may be used. The weight-average molecular weight is preferably 13,000,000 or less, more preferably, 10,000,000 or less, from the viewpoint of difficulty in synthesis.

For example, the Patent Document 1 (Japanese Laid-open Patent Publication No. 2002-233391) describes a PGA having a weight-average molecular weight of approximately 13,000,000, and the PGA can be used favorably in the present invention.

As described in the section of the background of the Patent Document 1, PGAs having a molecular weight of 100,000 to 2,000,000 have been commonly used and the PGAs having a molecular weight in the range also may be used in the present invention.

The PGA for use in the present invention may be produced by any known production method. For example, the method described in the aforementioned Patent Document 1 may be used.

### (Cholesterol or its Analogue)

The cholesterol or its analogue in the present invention include, in addition to unsubstituted cholesterol, substituted compounds carrying a substituent replacing a hydrogen atom bound to any carbon in the cholesterol molecule (i.e., hydrogen atom other than that in a hydroxyl group). Examples of the substituent include alkyl groups having about 1 to 5 carbon atoms, and the like. Cholesterol of which the unsaturated bond is hydrogenated is also included in the cholesterol or its analogue.

In addition, compounds similar to cholesterol having a chemical structure similar to that of cholesterol are also included in the cholesterol or its analogue according to the present invention. Examples of the substituted compounds include plant sterols, plant stanols, vitamin D group, provitamins and the like.

The plant sterol is, for example, β-sitosterol.

The plant stanol is, for example, β-sitostanol.

Examples of the vitamin D group include ergocalciferol (vitamin D2), cholecalciferol (vitamin D3), and 1,25-dihydroxy-cholecalciferol (activated vitamin D).

Examples of the provitamins include ergosterol and 7-dehydrocholesterol.

Cholesterol is a hydrophobic molecule having quite high biocompatibility that is also present in a human body, and it is characterized by having no primary nucleophilic substituent showing high reactivity with a carboxyl groups in PGA. The aforementioned cholesterol or its analogue having a substituent is also characterized by having no primary nucleophilic substituent group showing high reactivity with a carboxyl groups in PGA.

### (Linker)

In the present invention, the cholesterol or its analogue is bound to a side-chain carboxyl group of PGA via a linker.

Specifically, the PGA derivative is represented by the following Formula 2:

R¹-L-R² Formula 2

In the formula, R¹ represents a residue of PGA, L represents a linker; and R² represents a residue of cholesterol or its analogue.

The linker L is a bivalent group connecting the PGA in the PGA derivative to the cholesterol or its analogue. The molecular weight of the linker L is preferably 500 or less, and more preferably 300 or less. The molecular weight of the linker L is preferably 50 or more. The linker L is bound to the residue of the PGA side-chain carboxyl group at one end and to the residue of the hydroxyl group of the cholesterol at the other end.

The linker L may preferably have the structure represented by the following Formula 3: In such a case, the PGA derivative is represented by Formula 4.

—NH—R³—NHC(═O)— Formula 3

R¹—NH—R³—NHC(═O)—O—R² Formula 4

In the formulas, R³ is preferably an alkylene group, wherein the number of carbons is preferably 1 to 10, and more preferably 5 or less, and further more preferably 3 or less.

As for the moiety “C(═O)—O—” in the formula 3 above, if cholesterol chloroformate, for example, is used as the raw material for production of the PGA derivative, the moiety “C(═O)—O—” is formed from the chloroformate group.

The raw material for use in preparation of the moiety “—NH—R³—NH” in the linker represented by the above Formula 3 is preferably, for example, a diamino compound.

In the specification of the present application, a diamino compound refers to a compound having two amino groups. The amino group may be a primary amino group (—NH₂) or a secondary amino group (—NH—). A primary amino group is preferable.

Preferably, the diamino compound is alkylenediamine.

The number of carbons of the alkylenediamine is not particularly limited. The number of carbons of the alkylenediamine is preferably 2 or more. The number of carbons of the alkylenediamine is preferably 10 or less, more preferably 8 or less and further more preferably 6 or less. The alkylene group in the alkylenediamine may be a straight chain or a branched chain. It is preferably a straight chain.

Specifically, typical examples of the favorable alkylenediamines include 1,2-ethylenediamine, 1,3-propylenediamine, 1,4-butanediamine, 1,5-heptanediamine, 1,6-hexanediamine and the like.

Further, diamino compounds for use other than alkylenediamines include, for example, aromatic diamines. For example, 1,2-diaminobenzene, 1,3-diaminobenzene, 1,4-diaminobenzene, bis(4-aminophenyl)ether, and the like can be used.

### (Synthesis Method)

The method of preparing the compound according to the present invention is not particularly limited. However, in a preferable embodiment, the chloroformate of the cholesterol or its analogue is first obtained by a known method. The chloroformate of cholesterol or its analogue can be obtained, for example, by a method of reacting the cholesterol or its analogue with a large excess of phosgene. Further, cholesterol chloroformate and the like are commercially available as reagents. Therefore, such a commercially available reagent may be used. Subsequently, the chloroformate of cholesterol or its analogue and a diamino compound are mixed with each other, allowing reaction of the chloroformate of cholesterol or its analogue with one of the amino groups of the diamino compound to perform a dehydrochloric acid reaction, to form a cholesterolamine. The cholesterolamine and PGA are then reacted with each other, allowing the cholesterolamine to bind to the carboxyl groups of the PGA, to obtain cholesterol-introduced PGA. The reaction scheme is shown below:

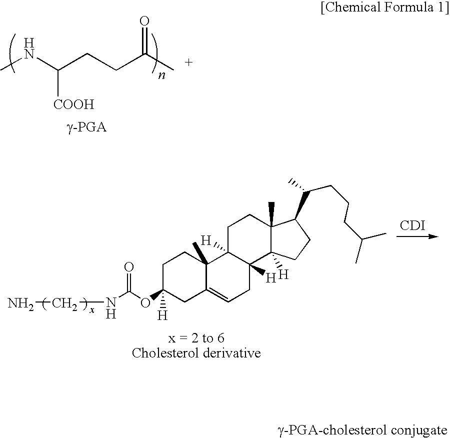

In the structural formula of the cholesterol derivative above, the structure of the moiety binding to the PGA is the structure derived from 1,2-ethylenediamine when X is 2, and the structure of the moiety binding to the PGA is the structure derived from 1,6-hexanediamine when X is 6.

(Reaction of Chloroformate of Cholesterol or its Analogue with Diamine)

Regarding a method of preparing cholesterolamine by a reaction of chloroformate of cholesterol or its analogue with a diamine, any known method for binding the chloroformate of cholesterol or its analogue to the amine may be used. For example, the method described in Langmuir 2001, 17, 5825-5833 may be used. In the method, cholesterol chloroformate is prepared from cholesterol or its analogue in advance, diamine is reacted thereto, and thereby cholesterolamine can be obtained.

The amount of the polyamine used is preferably, approximately 1 mole of polyamine with respect to 1 mole of the cholesterol or its analogue. The amount of the polyamine used is preferably 0.5 mole or more, more preferably 0.8 mole or more with respect to 1 mole of the cholesterol or its analogue. In addition, the amount of the polyamine used is preferably 2 moles or less, more preferably 1.5 moles or less, and further more preferably 1.2 moles or less, with respect to 1 mole of the cholesterol or its analogue.

The reaction between chloroformate of cholesterol or its analogue and the diamine does not particularly require a catalyst. However, but a catalyst known to be usable for a reaction between chloroformate and amine may be used as necessary.

Incidentally, in the specification of the present application, the compound in which the cholesterol or its analogue and a diamine are bound to each other that is obtained by the reaction of chloroformate of cholesterol or its analogue with the diamine, is also referred to as cholesterolamine.

(Reaction Between Cholesterolamine and PGA)

Examples of solvents for use in the reaction between cholesterolamine and PGA include dimethylsulfoxide, tetrahydrofuran and the like. In a preferable embodiment, PGA may be dispersed or dissolved in dimethylsulfoxide and the cholesterolamine may be dispersed or dissolved in tetrahydrofuran before use. When PGA has a high molecular weight, the PGA is likely to be less soluble in a solvent. However, the reaction can be carried out even if it is not dissolved completely in a solvent, i.e., even if it is dissolved only partially or dispersed in the solvent.

A dehydrating agent, which is known to be usable in a reaction between carboxylic acid and amine, may be used as necessary in the reaction between the cholesterolamine and PGA. Specifically, typical examples thereof include N,N′-carbonyldiimidazole (CDI), dicyclohexylcarbodiimide (DCC) and the like. Alternatively, water-soluble carbodiimides may also be used preferably. Typical examples of the water-soluble carbodiimides include 1-ethyl-3-(3-dimethylaminopropyl)carbodiimide (EDC) and the like.

The amount of CDI used is preferably 1 mole or more, more preferably 2 moles or more, and further more preferably 3 moles or more, with respect to 1 mole of the cholesterolamine. The amount of CDI used is also preferably 10 moles or less, more preferably 8 moles or less and further more preferably 6 moles or less with respect to 1 mole of the cholesterolamine.

Further, regarding a catalyst for the reaction between the cholesterolamine and PGA, a catalyst which is known to be usable for a reaction between carboxylic acid and amine may be used as necessary.

The temperature during the reaction is not particularly limited. The reaction may be carried out at room temperature or under heat. However, an excessively lower temperature may lead to significant elongation of the reaction, and thus, the reaction is preferably carried out at room temperature or higher. Specifically, it is carried out preferably at 10° C. or higher, more preferably at 15° C. or higher, and further more preferably at 20° C. or higher. It is also carried out preferably at 100° C. or lower, more preferably at 50° C. or lower. Excessively higher temperature may lead to easier decomposition of PGA. Thus, the reaction is preferably carried out at a temperature around room temperature.

The reaction period is preferably 30 minutes or longer, more preferably 1 hour or longer, further more preferably 2 hours or longer, yet further more preferably 6 hours or longer, and particularly preferably 12 hours or longer. However, for shortening of the period of time of the entire process, it is preferably 7 days or shorter, more preferably 4 days or shorter, and further more preferably 2 days or shorter.

After the reaction is complete, if necessary, the solvent is removed and purification is performed to obtain the PGA derivative according to the present invention.

(Product)

The PGA derivative obtained by the method described above has a structure in which cholesterolamine is bound covalently to some of the carboxyl groups in PGA. The cholesterolamine may be bound to all carboxyl groups in PGA. However, it is advantageous that cholesterolamine is bound covalently to only some of the carboxyl groups, since the properties of PGA are not deteriorated. The molar number of the cholesterolamine introduced can be controlled for example by adjusting the blending amounts and the reaction time in the reaction between the cholesterolamine and PGA. The molar number of the cholesterolamine introduced is appropriately designed depending on the application. The molar number of the cholesterolamine introduced is preferably 0.001% or more, more preferably 0.01% or more, further more preferably 0.1% or more, and particularly preferably 1% or more, with respect to the molar number of the side-chain carboxyl groups of the PGA before the reaction. The molar number of the cholesterolamine introduced is also preferably 20% or less, more preferably 15% or less, further more preferably 10% or less, yet further more preferably 5% or less, and particularly preferably 3% or less. When the amount of introduction is too small, the obtained PGA derivative is unlikely to form a fine particle. When the amount of introduction is too large, performance of the PGA in the obtained PGA derivative is likely to be deteriorated.

(Fine Particles)

The product obtained by the reaction described above may be placed in water and dispersed therein to form fine particles, with a process such as ultrasonication as necessary.

As will be explained in the Examples described below, the particle diameter of the fine particles can be determined for example by using a dynamic light-scattering detector (DLS). For example, in measurement by using a dynamic light-scattering detector under ultrasonication, the measured values become approximately constant within approximately 2 minutes. Therefore, the values at that time point may be used as the data of particle diameter.

Regarding the particle diameter, an average diameter is preferably 10,000 nm or less, more preferably 5,000 nm or less, and further more preferably 1,000 nm or less. The average diameter may be 700 nm or less, or 500 nm or less, in embodiments wherein a relatively small particle diameter is desirable. The average diameter is also preferably 5 nm or more, more preferably 10 nm or more, further more preferably 20 nm or more, and particularly preferably 50 nm or more. In embodiments wherein a relatively large particle diameter is desirable, it may be 100 nm or more or 200 nm or more. When the particle diameter is too large, dispersion stability is deteriorated, and further, properties as a nanoparticle are unlikely to be obtained. On the other hand, for a particle having a too small particle diameter, it is likely to be difficult to control production conditions.

Specifically, for example, a PGA derivative, which has 0.01 to 0.03 mole (i.e., 1% to 3%) of introduced cholesterol or its analogue with respect to 1 mole of the side-chain carboxyl groups of the PGA, gives particles having an average diameter of approximately 300 to 400 nm.

(Applications)

The PGA derivative according to the present invention is expected to have applications in a wide variety of fields. That is, since hydrophilicity is controlled while maintaining the biocompatibility of PGA, the PGA can be used as a carrier for various medicaments. Since the PGA can be formed into fine particles, it can be applied to various applications in the field of nanotechnology (e.g., DDS carrier and cell scaffold material).

(Sustained Release Carrier)

The PGA derivative according to the present invention may be used as a sustained release carrier of a medicament (e.g., DDS carrier). The carrier can be mixed with a pharmaceutically effective component to gives a sustained release pharmaceutical composition.

Nanoparticles consisting of an amphipathic molecule can contain a hydrophobic medicament. For this reason, the nanoparticles consisting of an amphipathic molecule are expected to be used as a DDS carrier. The PGA derivative according to the present invention is also useful as such a sustained release carrier of a medicament. Specifically, by combining the PGA derivative of the present invention with a pharmaceutically effective component, a sustained release pharmaceutical composition can be obtained. The PGA derivative of the present invention is useful, for example, as a sustained release carrier for vitamin D₃ (hereinafter, abbreviated as “VD”) obtained in biosynthetic pathway from steroid as starting material. In addition, if the PGA derivative according to the present invention is used as a sustained release carrier, the medicament used in combination is preferably a hydrophobic medicament.

The hydrophobic medicament refers to a medicament which is insoluble or slightly soluble in water. For example, the PGA derivative according to the present invention is useful for medicaments having a solubility of 5 mg/ml or less. Further the PGA derivative according to the present invention is useful for medicaments having a solubility of 1 mg/ml or less. There is no lower limit of the solubility and, for example, the solubility may be 0.01 mg/ml or more.

Any hydrophobic medicament conventionally known in the art may be used in the present invention. For example, the hydrophobic medicaments exemplified in Japanese National Phase Laid-open Patent Publication No. 10-503750 may be used. Typical examples thereof include the following medicaments:

Analgesics and anti-inflammatory agents: aloxiprin, auranofin, azapropazone, benorylate, diflunisal, etodolac, fenbufen, fenoprofen calcium, flurbiprofen, ibuprofen, indomethacin, ketoprofen, meclofenamic acid, mefenamic acid, nabumetone, naproxen, oxyphenbutazone, phenylbutazone, piroxicam, sulindac.

Anthelminthics: albendazole, bepheniun hydroxynaphthoate, cambendazole, dichlorophen, ivermectin, mebendazole, oxamniquine, oxfendazole, oxantel embonate, praziquantel, pyrantel embonate, thiabendazole.

Anti-arrhythmic agents: amiodarone HCl, disopyramide, flecamide acetate, quinidine sulphate.

Anti-bacterial agents: benethamine penicillin, cinoxacin, ciprofloxacin HCl, clarithromycin, clofazimine, cloxacillin, demeclocycline, doxycycline, erythromycin, ethionamide, imipenem, nalidixic acid, nitrofurantoin, rifampicin, spiramycin, sulphabenzamide, sulphadoxine, sulphamerazine, sulphacetamide, sulphadiazine, sulphafurazole, sulphamethoxazole, sulphapyridine, tetracycline, trimethoprim.

Anti-coagulants: dicoumarol, dipyridamole, nicoumalone, phenindione.

Anti-depressants: amoxapine, maprotiline HCl, mianserin HCl, nortriptyline HCl, trazodone HCl, trimipramine maleate.

Anti-diabetics: acetohexamide, chlorpropamide, glibenclamide, gliclazide, glipizide, tolazamide, tolbutamide.

Anti-epileptics: beclamide, carbamazepine, clonazepam, ethotoin, methoin, methsuximide, methylphenobarbitone, oxcarbazepine, paramethadione, phenacemide, phenobarbitone, phenyloin, phensuximide, primidone, sulthiame, valproic acid.

Anti-fungal agents: amphotericin, butoconazole nitrate, clotrimazole, econazole nitrate, fluconazole, flucytosine, griseofulvin, itraconazole, ketoconazole, miconazole, natamycin, nystatin, sulconazole nitrate, terbinafine HCl, terconazole, tioconazole, undecylenic acid.

Anti-gout agents: allopurinol, probenecid, sulphinpyrazone.

Anti-hypertensive agents: amlodipine, benidipine, darodipine, dilitazem HCl, diazoxide, felodipine, guanabenz acetate, isradipine, minoxidil, nicardipine HCl, nifedipine, nimodipine, phenoxybenzamine HCl, prazosin HCl, reserpine, terazosin HCl.

Anti-malarials: amodiaquine, chloroquine, chlorproguanil HCl, halofantrine HCl, mefloquine HCl, proguanil HCl, pyrimethamine, quinine sulphate.

Anti-migraine agents: dihydroergotamine mesylate, ergotamine tartrate, methysergide maleate, pizotifen maleate, sumatriptan succinate.

Anti-muscarinic agents: atropine, benzhexyl HCl, biperiden, ethopropazine HCl, hyoscyamine, mepenzolate bromide, oxyphencylcimine HCl, tropicamide.

Anti-neoplastic agents and Immunosuppressants: Aminoglutethimide, amsacrine, azathioprine, busulphan, chlorambucil, cyclosporin, Dacarbazine, estramustine, etoposide, lomustine, melphalan, mercaptopurine, methotrexate, mitomycin, mitotane, mitozantrone, procarbazine HCl, tamoxifen citrate, Testolactone, paclitaxel.

Anti-protozoal agents: benznidazole, Clioquinol, decoquinate, diiodohydroxyquinoline, diloxanide furoate, dinitolmide, furzolidone, metronidazole, nimorazole, nitrofurazone, ornidazole, tinidazole.

Anti-thyroid agents: carbimazole, propylthiouracil.

Anxiolytic, sedatives, hypnotics and neuroleptics: Alprazolam, amylobarbitone, barbitone, bentazepam, bromazepam, bromperidol, brotizolam, butobarbitone, carbromal, chlordiazepoxide, chlormethiazole, chlorpromazine, clobazam, clotiazepam, clozapine, diazepam, droperidol, ethinamate, flunanisone, flunitrazepam, fluopromazine, flupenthixol decanoate, fluphenazine decanoate, flurazepam, haloperidol, lorazepam, lormetazepam, medazepam, meprobamate, methaqualone, midazolam, nitrazepam, oxazepam, pentobarbitone, perphenazine pimozide, prochlorperazine, sulpiride, temazepam, thioridazine, triazolam, zopiclone.

Beta blocker: acebutolol, alprenolol, atenolol, labetalol, metoprolol, nadolol, oxprenolol, pindolol, propranolol.

Cardiac Inotropic agents: aminone, digitoxin, digoxin, enoximone, lanatoside, medigoxin.

Corticosteroids: beclomethasone, betamethasone, budesonide, Cortisone acetate, desoxymethasone, dexamethasone, fludrocortisone acatate, flunisolide, flucortolone, fluticasone propionate, hydrocortisone, methylprednisolone, prednisolone, prednisone, triamcinolone.

Diuretics: acetazolamide, amiloride, bendrofluazide, bumetanide, chlorothiazide, chlorthalidone, Ethacrynic acid, frusemide, metolazone, spironolactone, Triamterene.

Anti-parkinsonian agents: bromocriptine mesylate, Lysuride maleate.

Gastro-intestinal agents: bisacodyl, cimetidine, cisapride, diphenoxylate HCl, domperidone, famotidine, loperamide, mesalazine, nizatidine, omeprazole, Ondansetron HCl, ranitidine HCl, sulphasalazine.

Histamine H-Receptor Antagonists: acrivastine, astemizole, cinnarizine, cyclizine, cyproheptadine HCl, dimenhydrinate, flunarizine HCl, loratadine, meclozine HCl, oxatomide, terfenadine.

Lipid regulating agents: bezafibrate, clofibrate, fenofibrate, gemfibrozil, probucol.

Nitrates and anti-anginal agents: amyl nitrate, glyceryl trinitrate, isosorbide dinitrate, isosorbide mononitrate, pentaerythritol tetranitrate.

Nutritional agents: beta-carotene.

Lipid soluble vitamins: vitamin A, vitamin B₂, vitamin D, vitamin E, vitamin K.

Opioid analgesics: codeine, dextropropyoxyphene, Diamorphine, dihydrocodeine, meptazinol, methadone, morphine, nalbuphine, pentazocine.

Sex hormones: clomiphene citrate, danazol, ethinyloestradiol, medroxyprogesterone acetate, mestranol, methyltestosterone, norethisterone, norgestrel, oestradiol, conjugated oestrogens, progesterone, stanozolol, stiboestrol, testosterone, tibolone.

Stimulants: amphetamine, dexamphetamine, dexfenfluramine, fenfluramine, mazindol.

In one embodiment, the blending amount of a pharmaceutically effective component in a pharmaceutical composition is 0.0001% by weight or more, preferably 0.001% by weight or more, more preferably 0.01% by weight or more, and further more preferably, 0.1% by weight or more. In an embodiment where the pharmaceutically effective component is blended in a greater amount, the amount is, for example, 1% by weight or more. Further in one embodiment, the amount is 50% by weight or less, preferably 20% by weight or less, more preferably 10% by weight or less. In an embodiment where it is desirably blended in a small amount, the amount is, for example, 5% by weight or less. The amount may be 1% by weight or less as necessary.

In one embodiment, the blending amount of the PGA derivative according to the present invention in a pharmaceutical composition is 1% by weight or more, preferably 10% by weight or more, more preferably 30% by weight or more and further more preferably 50% by weight or more. In an embodiment where it is desired that a greater amount is blended, the amount may be, for example, 70% by weight or more, 80% by weight or more, or 90% by weight or more. Further, in an embodiment, the amount is 99% by weight or less, preferably 95% by weight or less, and more preferably, 90% by weight or less. In an embodiment where it is desirable that a relatively small amount is blended, the amount may be, for example, 80% by weight or less, 70% by weight or less, 60% by weight or less, or 50% by weight or less. The amount may be 1% or less, if necessary.

The pharmaceutical composition may consist of only the PGA derivative according to the present invention and a pharmaceutically effective component. The pharmaceutical composition may contain other additives and the like, if necessary. Examples of other additives which may be added to the pharmaceutical composition include carriers other than the PGA derivatives according to the present invention, colorants, and the like.

The pharmaceutical composition may be administered in any dosage form. It is preferably in a solid form. The solid form includes a tablet, capsule, powder, and the like. Further, it is possible to use the composition in a liquid form.

(Molecular Chaperone)

The PGA derivative according to the present invention may be used as a molecular chaperone for folding a denatured protein into a correct steric structure. Herein, “denaturation” includes denaturation by physical factors such as heat and denaturation by chemical factors such as a modifying agent. Further, the folding into a correct steric structure is also referred to as refolding.

The PGA derivative of the present invention may be used as a molecular chaperone for refolding denatured proteins in the non-aggregated state (solution state) or as a molecular chaperone for refolding denatured proteins in the aggregated state. Specifically, the PGA derivative of the present invention can act as a refolding agent for protein in the denatured state, and can also act as a refolding agent for protein in the aggregated state.

When the PGA derivative of the present invention is used as a molecular chaperone, the type of the protein is not particularly limited. The present invention is useful for proteins exerting biologically functions. In particular, the PGA derivative of the present invention is useful for processing proteins that exert a certain function in a certain steric structure but can not exert the function when the steric structure is altered. Regarding a type of such a protein, preferable examples include enzymes, protein drugs (cytokines, various growth factors, antigen proteins, etc.), and the like.

The molecular weight of the protein is preferably 1,000 or more, and more preferably 3,000 or more. In an embodiment, the molecular weight of the protein may be 10,000 or more. The molecular weight of the protein is preferably 10,000,000 or less, and more preferably 5,000,000 or less. In an embodiment, the molecular weight of the protein may be 1,000,000 or less.

Examples of the aforementioned enzymes include oxidoreductases, transferases, hydrolases, lyases, isomerases, synthases, and the like. Examples of the oxidoreductases include glucose oxidase, catalase, lipoxygenase, and the like. Examples of the transferases include amino group transferase, glycosyl transferase, DNA polymerase, phosphorylase, and the like. Examples of the hydrolases include amylase, protease, lipase, lysozyme, and the like. Examples of the lyases include alginic acid lyase, carbonate dehydrogenase, citric acid synthase, and the like. Examples of the isomerases include xylose isomerase, phosphoglucomutase, and the like. Examples of the synthases include DNA ligase, glutamine synthase, and the like.

Examples of the aforementioned cytokines include interleukins, interferons, lymphotoxins, colony stimulus factors, tumor necrosis factors, and the like.

Examples of the aforementioned growth factors include epidermal growth factor (EGF), insulin-like growth factor (IGF), transforming growth factor (TGF), nerve growth factor (NGF), brain-derived nerve nutritional factor (BDNF), vascular endothelial cell growth factors (VEGF), granulocyte colony stimulus factor (G-CSF), granulocyte macrophage colony stimulus factor (GM-CSF), platelet-derived growth factor (PDGF), erythropoietin (EPO), thrombopoietin (TPO), basic fibroblast growth factor (bFGF or FGF2), hepatocyte growth factor (HGF), and the like.

Examples of the aforementioned antigen proteins include various tumor-related proteins, various infection antigen proteins (influenza, avian influenza, AIDS, typhoid, dysentery, cholera, etc.), and the like.

When the PGA derivative of the present invention is used as a molecular chaperone, the protein refolding may be performed, for example, in the following manner:

A denatured protein (e.g., enzyme) is brought into contact with a PGA derivative. For example, a solution of the PGA derivative is prepared. Separately, a solution of the denatured protein is prepared. The PGA derivative solution and the denatured protein solution are mixed to prepare a complex of the PGA derivative and the protein. When the complex of the PGA derivative and the protein is formed, aggregation of the protein can be suppressed.

In the case where denatured protein is in the aggregated state and it is difficult to prepare a solution thereof, the PGA derivative solution may be added to the aggregate without preparing a solution.

Removal of the PGA derivative from the complex of the PGA derivative and the protein gives a refolded protein. As a method of removing the PGA derivative from the complex of the PGA derivative and the protein, for example, use of a method of adding cyclodextrin (e.g., alpha-CD, beta-CD or gamma-CD) to the complex of the PGA derivative and the protein leads to unpacking of cholesterol groups, releasing a refolded protein.

### EXAMPLES

Hereinafter, Examples of a method of introducing a cholesterol derivative to PGA will be described.

### (Preparation of Cholesterol Derivatives)

The cholesterol derivative prepared is represented by the following General Formula 1:

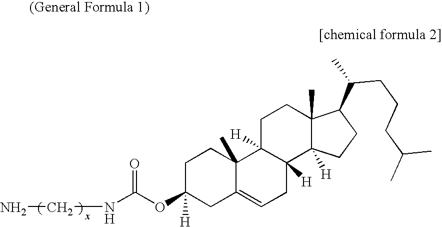

The cholesterol derivative represented by the General Formula 1 was prepared according to the method described in Langmuir 2001, 17, 5825-5833 in the following manner:

Molecular sieve 3A (50 g) which was dried at 10° C. for 1 hour was added to toluene (500 mL). It was left stand overnight, to prepare dehydrated toluene. Ethylenediamine (16.7 mL, 250 mmol) was dissolved in the dehydrated toluene (250 mL) and placed in a three-necked flask, and the container was kept at 0° C. in an ice bath in nitrogen atmosphere. Cholesterol chloroformate (2.25 g, 5 mmol) was dissolved in dehydrated toluene (50 mL), the solution was placed in a dropping funnel and set on the three-necked flask, and then, the liquid was stirred while maintaining the reaction container at 0° C. under a nitrogen atmosphere, and the cholesterol chloroformate solution was added dropwise over 10 minutes. After completion of the dropping, the temperature was allowed to return to room temperature and reaction was conducted for 16 hours. After completion of the reaction, the solution was transferred into a separatory funnel. The raw material, ethylenediamine, was sufficiently extracted with deionized water. The organic (toluene) layer was dried over anhydrous magnesium sulfate. The organic layer obtained was concentrated under reduced pressure in an evaporator, to give a crude solid product, which was then washed with a mixed solvent of dichloromethane (20 mL) and methanol (20 mL). The produced suspension was filtered for removal of insoluble dicarbamate (di-substituted), and the filtrate was concentrated again in an evaporator under reduced pressure, to give a desired white solid product (mono-substituted) (yield 70%).

(Preparation of PGA)

PGA having a molecular weight of approximately 50,000 was prepared according to the method described in International Publication WO 2004-7593. It was used in the following experiments:

### Example 1

The aforementioned PGA in an amount equivalent to 3 mmol as a glutamic acid monomer was dissolved in 10 mL of dimethylsulfoxide (DMSO).

Further, 0.03 mmol of the aforementioned cholesterolamine (CHAm) was dissolved in 10 mL of tetrahydrofuran (THF).

The aforementioned PGA solution and the cholesterolamine solution were then mixed with each other. Then, 0.12 mmol of N,N′-carbonyldiimidazole (CDI) was added thereto, and the mixture was stirred at room temperature for 24 hours. After completion of the reaction, THF was removed from the reaction product in a reduced-pressure concentrating apparatus (evaporator), and then rough purification was performed by a reprecipitation process using toluene. Toluene was removed from the rough purification product, and thereafter the resulting rough purification product was neutralized with sodium bicarbonate. The aqueous solution was purified by dialysis using a dialysis membrane having a molecular weight cut-off of 2,000 for 2 days (exchange of water, 4 times). The content was then freeze-dried to give a product. The yield with respect to the amounts of the PGA and the cholesterolamine derivative used (theoretical yield calculated by assuming that the CHAm used was all introduced into the used PGA) was 91%.

Synthesis of the desired product was confirmed with the nuclear magnetic resonance spectrum. As a result, the hydrogen peaks of the unsaturated moiety in the cholesterol skeleton (5.38 ppm) was significantly broadened in comparison with that before binding to PGA. Simultaneously, the position of the peak was slightly shifted to the side of the lower magnetic field, and the peak appeared around 5.44 ppm. Therefore, it was confirmed that cholesterolamine was bound to the side chains of PGA. In addition, it was observed that the peaks derived from the cholesterol residue observed in the region at around 0.65 ppm to 1.48 ppm were also significantly broadened. Therefore, it was confirmed that cholesterolamine was bound to the side chain of the PGA.

As for the cholesterol introduction ratio, as a result of the analysis of NMR of the obtained PGA derivative, from the ratio of the integrated value of the peak of the hydrogen bound to the carbon at the beta position in the PGA residue (4.1 ppm) and integrated value of the peak of the hydrogen bound to the unsaturated moiety in the cholesterol skeleton (5.4 ppm), it was confirmed that cholesterol was introduced to approximately 1.1% of the side-chain carboxyl groups of the PGA.

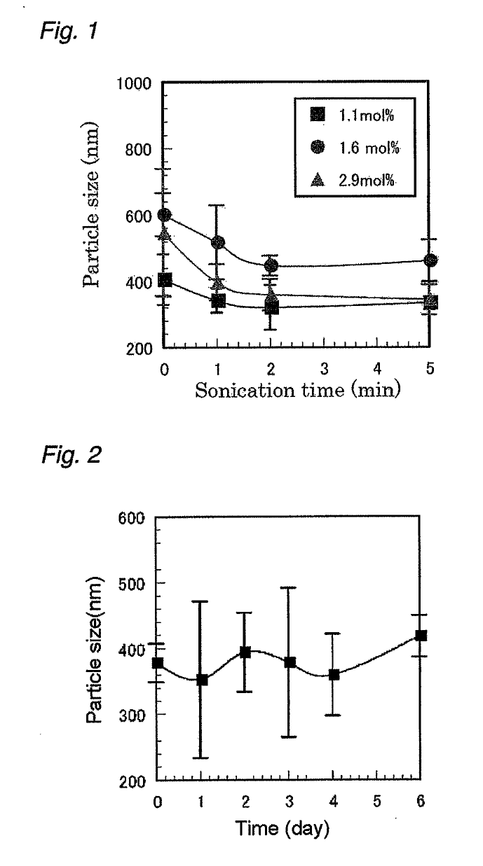

A 0.05 wt % aqueous solution of the PGA derivative obtained was prepared and processed in a homogenizer for a certain period of time, and then, a measurement of a particle diameter of the formed fine particle was performed using a dynamic light-scattering detector (DLS). The data became constant after ultrasonication for about 2 minutes, and a particle diameter of about 330 nm on average was measured. The results are shown in FIG. 1.

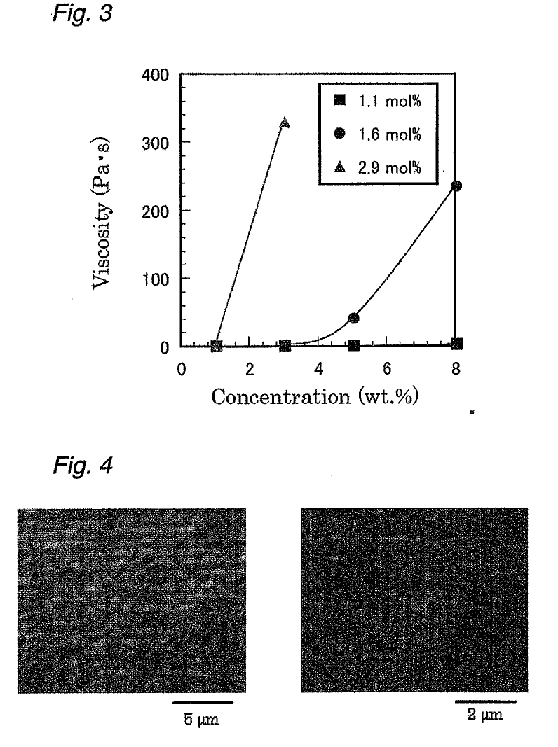

In addition, the concentration dependence of the viscosity of the aqueous solution of the PGA derivative obtained was confirmed by performing measurement of viscosity. Even though the concentration was increased to 8%, the viscosity was not increased (FIG. 3).

Then, analysis under SEM (scanning electron microscope) of the particles obtained by drying the aqueous solution used in the aforementioned particle diameter measurement was performed. Particles having a diameter of several hundreds nm were confirmed. The images obtained are shown in FIG. 4.

### Example 2

A test was carried out in a manner similar to Example 1, except that 0.075 mmol of the aforementioned cholesterolamine derivative (CHAm) and 0.3 mmol of the N,N′-carbonyldiimidazole (CDI) were used. As a result, the yield based on the amounts of the PGA and the cholesterolamine derivative used (theoretical yield calculated by assuming that the CHAm used was all introduced into the PGA) was 68%. The cholesterol introduction ratio was 1.6%.

A 0.05 wt % aqueous solution of the derivative obtained was prepared and processed in a homogenizer for a certain period of time; and particle diameter measurement of the formed fine particles was performed by using a dynamic light-scattering detector (DLS). The data became constant after ultrasonication for approximately 2 minutes, and an average particle diameter of approximately 460 nm was measured. The results are shown in FIG. 1.

Further, the aqueous solution was left stand for 6 days, and the change in diameter of the fine particles during the term was observed (FIG. 2).

The viscosity of the aqueous solution of the derivative obtained was also determined. As a result, it was confirmed that the viscosity increased in a concentration range of 5% or more (FIG. 3).

Further, 10 mg of β-cyclodextrin (β-CD) was added to 1 mL of an 8% aqueous solution of the obtained derivative, and viscosity measurement was carried out. As a result, the viscosity decreased significantly to approximately 10 Pa·s. It is considered that the viscosity was decreased because the cholesterol residue was included in the β-CD and thereby the hydrophobic interaction among cholesterol residues is decreased.

### Example 3

A test was carried out in a manner similar to the aforementioned Example 1, except that 0.15 mmol of the aforementioned cholesterolamine derivative (CHAm) and 0.6 mmol of the N,N′-carbonyldiimidazole (CDI) were used. The resulting cholesterol introduction ratio was 2.9%.

A 0.05 wt % aqueous solution of the obtained derivative was prepared and processed in a homogenizer for a certain period of time, and particle diameter measurement of the formed fine particle was performed by using a dynamic light-scattering detector (DLS). The data became constant after ultrasonication for approximately 2 minutes, showing that the average particle diameter was approximately 340 nm. The results are shown in FIG. 1.

The viscosity of the aqueous solution of the derivative obtained was also determined. As a result, it was confirmed that the viscosity increased in a concentration range of 5% or more (FIG. 3).

The conditions and the results in Examples 1 to 3 are shown in the following Table.

TABLE 1

| | CHAm/PGA | Yield | Introduction ratio |
|---|---|---|---|
| Example | (mol %) | (%) | (mol %) |
| 1 | 1 | 91 | 1.1 |
| 2 | 2.5 | 68 | 1.6 |
| 3 | 5 | — | 2.9 |

### Example 4

A test was carried out in a manner similar to the aforementioned Example 2, except that a PGA having a molecular weight of approximately 2,000,000 was used and the rough purification was performed by reprecipitation using water, instead of the reprecipitation using toluene. As a result, the yield based on the amounts of the PGA and the cholesterolamine derivative used (theoretical yield calculated by assuming that the CHAm used was all introduced into the PGA) was 6.1%. The cholesterol introduction ratio was 2.7%.

### Example 5

A test was carried out in a manner similar to the aforementioned Example 1, except that 0.06 mmol of the cholesterol derivative (CHAm) and 0.24 mmol of N,N′-carbonyldiimidazole (CDI) were used. The resulting yield based on the amounts of the PGA and the cholesterolamine derivative used (theoretical yield calculated by assuming that the CHAm used was all introduced into the PGA) was 85%. The cholesterol introduction ratio was 1.3%.

### Examples 6 and 7

Tests were carried out in a manner similar to Example 5, except that the conditions were altered as shown in the following Table 2.

### Example 8

A test was carried out in a manner similar to the aforementioned Example 1, except that 1,6-hexanediamine was used in place of ethylenediamine in preparation of the cholesterol derivative described above, and 0.051 mmol of the cholesterol derivative (Chemical Formula 1, X=6) and 0.24 mmol of CDI were used. The resulting yield based on the amounts of the PGA and the cholesterol derivative used (theoretical yield calculated by assuming that the CHAm used was all introduced into the PGA) was 72%. The cholesterol introduction ratio was 0.84%.

### Examples 9 to 10

Tests were carried out in a manner similar to Example 8, except that the conditions were altered as shown in the following Table 2.

The results obtained in Examples 5 to 10 are shown in the following Table 2. In the Table, “X” in the columns of the cholesterol derivative represents X in the above-described General Formula.

TABLE 2

| | | cholesterol | | CHAm/ | | | Introduction |
|---|---|---|---|---|---|---|---|
| Exam- | | derivative | | PGA | CDI | Yield | Ratio |
| ple | Sample | X | [mmol] | [mol %] | [mmol] | [%] | [%] |
| 5 | CHE 2 | 2 | 0.06 | 2 | 0.24 | 85 | 1.3 |
| 6 | CHE 3 | 2 | 0.09 | 3 | 0.36 | 72 | 1.5 |
| 7 | CHE 4 | 2 | 0.15 | 5 | 0.6 | 73 | 2.1 |
| 8 | CHH 1 | 6 | 0.051 | 1.7 | 0.24 | 72 | 0.84 |
| 9 | CHH 2 | 6 | 0.075 | 2.5 | 0.36 | 65 | 0.97 |
| 10 | CHH 3 | 6 | 0.126 | 4.3 | 0.6 | 77 | 2.1 |

### Example 11

### Evaluation of Sustained Release Efficiency of a Medicament

The Sustained Release Efficacy of the Derivative According to the Present invention was evaluated, by using vitamin D₃ (hereinafter, abbreviated as “VD”) obtained in the biosynthetic pathway from steroid as a starting material.

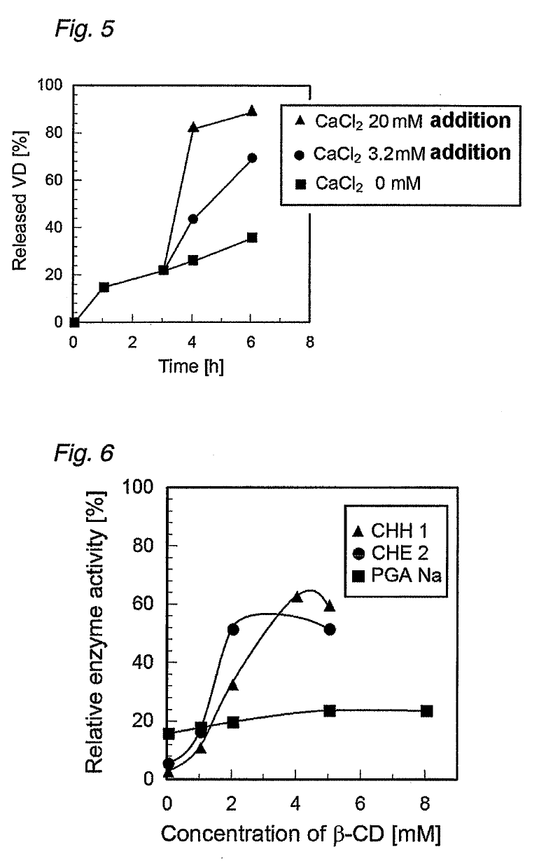

The CHH 1 (10 mg) obtained in Example 1 described above was dissolved in phosphate-buffered physiological saline (10 mL). Further, VD (10 mg) was dissolved in ethanol (10 mL). The two solutions were mixed, and ethanol was distilled off by evaporation. At that time, some water was vaporized. Therefore, deionized water was added such that the total amount of the solution is 10 g. The solution obtained was centrifuged at 12,000 rpm for 30 minutes, and the supernatant solution (1 mL) was collected. The solution was evaporated such that water was vaporized. VD was extracted from the solid obtained from ethanol, and the UV spectrum thereof was measured. The amount of VD in the supernatant liquid was determined by comparing the intensity of the peaks in the UV spectrum obtained with the intensity of the peaks of samples containing a known amount of VD. The amount of VD obtained in the measurement was designated as an amount of initial VD inclusion. The other solutions were shaken at 37° C. for a certain period of time (1, 3, 4 or 6 hours), a similar operation was performed, to measure an amount of released VD at each of the time points. The results are shown in the graph of FIG. 5, as indicated by black square symbols. In the graph of FIG. 5, the abscissa indicates the time elapsed after the collection of the “initial” sample, while the ordinate represents the released VD (%). The ratio of the released VD was calculated according to the following calculation formula:

Released VD (%)=Wₜ/W₀×100

W₀: weight of initial VD
Wₜ: weight of VD at time t

As shown in the graph, the amount of the released VD at the time point of 1 hour was approximately 16%; the amount of the released VD at the time point of 3 hours was approximately 20%; the amount of the released VD at the time point of 4 hours was approximately 24%; and the amount of the released VD at the time point of 6 hours was approximately 36%. That is, it was confirmed that VD was released gradually over a long period of time. It was confirmed that there are excellent properties as a sustained release carrier. Accordingly, the nanoparticles obtained can be used effectively as a DDS carrier for a medicament having a steroid skeleton.

### Example 12

### Evaluation of Sustained Release Efficiency of a Medicament

A test similar to Example 11 was carried out. However, 3.2 mM of CaCl₂ was added at the time point of 3 hours. The results are shown with circular symbols in the graph of FIG. 5.

As a result, the amount of released VD at the time point after 4 hours was approximately 40%, and the amount of released VD at the time point after 6 hours was approximately 68%. That is, it was confirmed that VD was rapidly released after the addition of calcium chloride.

### Example 13

### Evaluation of Sustained Release Efficiency of a Medicament

A test similar to Example 12 was carried out. However, 20 mM of CaCl₂ was added at the time point after 3 hours. The results are shown with triangular symbols in the graph of FIG. 5.

As a result, the amount of released VD at the time point after 4 hours was approximately 82%, and the amount of Released VD after 6 hours was approximately 90%. That is, it was confirmed that VD was released quite rapidly after addition of calcium chloride.

The calcium ion response shown by the results in Examples 12 and 13 is a characteristic quite rarely found in conventional sustained release carriers. It is possible to control the medicament release rate from the derivative according to the present invention by adjusting an amount of calcium. Therefore, the derivative according to the present invention is extremely useful as a sustained release carrier.

### Example 14

The derivative according to the present invention can be used as a molecular chaperone. In the present Example, the properties of the derivative according to the present invention as a refolding agent were confirmed.

A carbonic anhydrase, Bovine carbonic anhydrase (BCA) (purchased from SIGMA (product number: C3934)) (3 mg) and urea (60 mg) were dissolved in 50 mM Tris-sulfate buffer (pH 7.5, 100 mL), and the solution was left stand at 25° C. for 16 hours, to cleave hydrogen bonds of the BCA, and thereby a denatured BCA was obtained.

The PGA derivative obtained in Example 5 described above was dissolved in a buffer solution (50 mM Tris-Sulfate buffer (pH 7.5)) (5.0 mg/mL). The denatured BCA (10 μl) was diluted 1,000 times with the sample solution (10 mL), to give a diluted solution. The complex of the PGA derivative and the protein was formed in the diluted solution obtained. The dilute solution was shaken at 37° C. for approximately 2 hours. A predetermined amount of β-CD was then added thereto, and the mixture was left stand at 37° C. for 24 hours.

The amount of reacted p-nitrophenyl acetate (pNPA), which was the substrate, was measured, such that the activity of the obtained enzyme solution was measured, and thereby the refolding ratio was calculated. Specifically, 1 mL of the solution obtained (BCA final concentration: 0.03 mg/mL) and 10 μl of pNPA dissolved in acetonitrile (100 mM) were mixed with each other, and the UV measurement of the mixture was performed (400 nm, 60 s). The difference between the absorbance at time point of 30 s and the absorbance at time point of 40 s was calculated, and thereby the enzyme activity was evaluated.

In addition, the activity of the enzyme before denaturation was evaluated as comparative data. The relative enzyme activity (%) was calculated according to the calculation formula below, from the ratio of the enzyme activity after the aforementioned refolding experiment to the enzyme activity before denaturation. The value obtained by this calculation shows the recovery ratio of the enzyme activity, and thus, the value was evaluated as the refolding ratio.

$$Relative enzyme activity ( \% ) = \frac{Δ {Abs}_{400 nm} ( sample )}{Δ {Abs}_{400 nm} ( native CAB )} \times 100 \qquad [ Chemical formula 3 ]$$

As a result, the refolding ratio, i.e., the relative enzyme activity, was approximately 52% when 2 mM of β-CD was used. It was approximately 50% when 5 mM of β-CD was used. The enzyme activity was increased depending on the amount of the added β-CD. Therefore, it was confirmed that a refolded enzyme was released from the complex of the PGA derivative and the protein.

### Example 15

A test was carried out in a manner similar to Example 14, except that the PGA derivative obtained in Example 8 (5.0 mg/mL) was used instead of the PGA derivative obtained in Example 5.

As a result, the refolding ratio, i.e., the relative enzyme activity, was approximately 32% when 2 mM of β-CD was used. It was approximately 62% when 4 mM of β-CD was used. It was approximately 60% when 5 mM of β-CD was used. The enzyme activity was increased depending on the amount of added β-CD. Therefore, it was confirmed that a refolded enzyme was released from the complex of the PGA derivative and the protein.

### Comparative Example 1

A test was carried out in a manner similar to Example 14, except that the PGANa (5.0 mg/mL) was used instead of the PGA derivative obtained in Example 5. As a result, the enzyme was aggregated in the diluted solution. The refolding ratio, i.e., the relative enzyme activity, was approximately 20% when the amount of the added β-CD was 0 to 8 mM.

### Comparative Example 2

A test was carried out in a manner similar to Example 14, except that the PGA derivative obtained in Example 5 was not used. As a result, the enzyme was aggregated in the diluted solution. The refolding ratio, i.e., the relative enzyme activity, was approximately 20% when the amount of the added β-CD was 0 to 8 mM.

The results obtained in Examples 14 and 15 and Comparative Example 1 are shown in FIG. 6. The abscissa in FIG. 6 indicates the amount of β-CD (mM) added, and the ordinate indicates the relative activity (%) of the enzyme.

As a result, the refolding ratio was approximately 20% when no sample was added or when PGA sodium salt (PGANa) was used, while a higher refolding ratio was shown when the PGA derivative in Example 5 (CHE 2) or the PGA derivative in Example 8 (CHH 1) was used. That is, it was confirmed that the PGA derivative according to the present invention could act as a molecular chaperone of refolding proteins.

### Example 16

A sample of denatured enzyme was prepared by cleaving the hydrogen bonds in carbonic anhydrase (3 mg) in a manner similar to Example 14. The modified BCA sample obtained was diluted with a buffer solution (50 mM Tris-sulfate buffer (pH 7.5)), and the resulting precipitate was used as the enzyme aggregate (inclusion body model). A solution of the PGA derivative obtained in Example 8 (5.0 mg/mL) was prepared in a manner similar to Example 14. The aggregate was added to the solution. The container was shaken at 37° C. for approximately 2 hours. Then, a predetermined amount of β-CD was added, and the mixture was left stand at 37° C. for 24 hours. The activity of the enzyme solution obtained was determined in a similar manner to Example 14.

As a result, the refolding ratio, i.e., the relative enzyme activity, was approximately 2% when 1 mM of β-CD was used. It was approximately 20% when 2 mM of β-CD was used. It was approximately 42% when 5 mM of β-CD was used. The enzyme activity was increased depending on the amount of the added β-CD. Therefore, it was confirmed that a refolded enzyme was released.

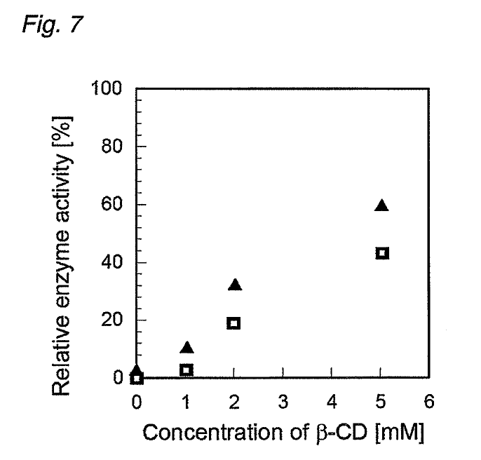

Results are shown in FIG. 7. The abscissa in FIG. 7 indicates the amount of the added β-CD (mM), and the ordinate indicates the relative activity (%) of the enzyme. The results obtained in Example 16 are indicated by white square symbols. The results obtained in Example 15 are indicated by triangular symbols.

As a result, it was confirmed that, even if the PGA derivative was added after a protein was aggregated, the PGA derivative has high refolding efficiency.

As described above, the present invention has been illustrated using the preferred embodiments of the present invention. However, the present invention should not be construed to be limited to the embodiments. It is understood that the scope of the present invention should be construed solely on the basis of the claims. It is understood that those skilled in the art can carry out an invention within a scope, which is equivalent to the description of the specification, based on the description of the specific preferred embodiments, the description of the present invention and the common technical knowledge. It is understood that the patents, patent applications, and other documents cited in the present specification should be incorporated by reference in the present specification as if the contents thereof are specifically described herein.

### INDUSTRIAL APPLICABILITY

The present invention provides a PGA derivative whose hydrophilicity is appropriately controlled. Further, the present invention provides a PGA derivative that can be processed into a form of fine particles. Therefore, it is made possible to mix the PGA derivative with various biological materials (e.g., medicaments) and to use the mixture in various applications in the field of biotechnology. Specifically, it is made possible to use the PGA derivative as a carrier of medicaments and the like. It is also made possible to use the PGA derivative in various applications in the field of nanotechnology such as a DDS carrier, and cell scaffold material.

1. A poly-γ-glutamic acid derivative, wherein cholesterol or its analogue is bound to the poly-γ-glutamic acid via a linker.

2. The poly-γ-glutamic acid derivative according to claim 1, which is represented by the following Formula 1:
R¹—NH—R³—NHC(═O)—O—R² Formula 1
wherein R¹ represents a residue of the poly-γ-glutamic acid, —O—R² represents a residue of the cholesterol or its analogue, and R³ represents alkylene.

3. The poly-γ-glutamic acid derivative according to claim 1, wherein a weight-average molecular weight of the poly-γ-glutamic acid is 10,000 or more.

4. The poly-γ-glutamic acid derivative according to claim 1, wherein the cholesterol or its analogue is bound to 0.1% to 10% of the side-chain carboxyl groups of the poly-γ-glutamic acid.

5. A fine particle having an average diameter of 50 nm to 1,000 nm, consisting of the poly-γ-glutamic acid derivative according to claim 1.

6. A method for producing the fine particle according to claim 5, comprising a step of introducing cholesterol or its analogue into poly-γ-glutamic acid.

7. A molecular chaperone for refolding a protein in a denatured state, consisting of the poly-γ-glutamic acid derivative according to claim 1.

8. A molecular chaperone for refolding a denatured protein in an aggregation state, consisting of the poly-γ-glutamic acid derivative according to claim 1.

9. A sustained release carrier, consisting of the poly-γ-glutamic acid derivative according to claim 1.
